(19) United States
(12) Patent Application Publication
(10) Pub. No.: US 2006/0160852 A1
(43) Pub. Date: Jul. 20, 2006

(54) Composition containing anti-dementia drug

(75) Inventors: Susumu Kimura, Kakamigahara-shi (JP); Yosuke Ueki, Kakamigahara-shi (JP); Satoshi Fujioka, Kakamigahara-shi (JP)

Correspondence Address: VENABLE LLP, P.O. BOX 34385, WASHINGTON, DC, 20045-9998, US

(73) Assignee: Eisai Co. LTD., Tokyo (JP)

(21) Appl. No.: 11/317,238

(22) Filed: Dec. 27, 2005

Related U.S. Application Data
(60) Provisional application No. 60/675,483, filed on Apr. 28, 2005.

(30) Foreign Application Priority Data
Dec. 27, 2004 (JP) 2004-376770

(51) Int. Cl. A61K 31/445; A61K 31/13

(52) U.S. Cl. 514/319; 514/663

(57) ABSTRACT

An object of the present invention is to provide, for the case of implementing a therapeutic method in which at least two kinds of anti-dementia drugs are used together, a composition that has a good therapeutic effect on the dementia, and also gives excellent compliance. Another object of the present invention is to provide a composition containing at least two kinds of the anti-dementia drugs, for which release of the anti-dementia drugs from the composition is controlled, whereby a combined effect of the anti-dementia drugs can be achieved well. Still another object of the present invention is to provide: a composition, for which the frequency of administration and the amount taken are reduced, and hence compliance can be improved; and a method of manufacturing such a composition. According to the present invention, there are provided a composition containing at least two kinds of the anti-dementia drugs; such a composition containing at least one sustained release portion containing an anti-dementia drug; and such a composition containing at least one cholinesterase inhibitor, and at least one N-methyl-D-aspartate receptor antagonist.

### BACKGROUND OF INVENTION

1. Field of the Invention

The present invention relates to a composition containing an anti-dementia drug. More particularly, the present invention relates to a composition containing at least two kinds of anti-dementia drugs.

2. Description of the Related Art

In recent years, care for dementia such as senile dementia and Alzheimer-type dementia has become a social problem, and many therapeutic drugs for dementia are being developed. Of these, donepezil, which has been supplied as the hydrochloride in a tablet or a granule form (trade name Aricept, manufactured by Eisai Co., Ltd.), is seen as being highly useful as a therapeutic drug for mild to moderate Alzheimer-type dementia due to having an acetylcholinesterase inhibiting action. Moreover, memantine hydrochloride, which exhibits antagonism towards N-methyl-D-aspartate (NMDA) receptors, has also been developed as a therapeutic drug for moderate to severe Alzheimer-type dementia, and has been supplied in a film-coated tablet or liquid form (trade name Axura, manufactured by Merz Pharmaceuticals; trade name Namenda, manufactured by Forest Pharmaceuticals, Inc.).

Recently, trials have been made using these two drugs together. It has been reported that upon further administering memantine hydrochloride or a placebo using a double blind test method to patients with moderate to severe Alzheimer-type dementia who had been administered donepezil hydrochloride, for the group administered both donepezil hydrochloride and memantine hydrochloride, cognitive ability and activities of dairy living were improved as compared to the group administered the placebo (See Pierre N. Tariot et al., “Memantine Treatment in Patients with Moderate to Severe Alzheimer Disease Already Receiving Donepezil—a Randomized Controlled Trial”, JAMA, Vol. 291, No. 3, p. 317-324). Moreover, the idea of a preparation containing an acetylcholinesterase inhibitor and an NMDA receptor antagonist has also been disclosed (See International Publication No. 03/101458, and U.S. Patent Application Publication No. 2004/0087658).

Meanwhile, most Alzheimer-type dementia patients not only have reduced cognitive ability, but also have difficulty in swallowing, and so sufficient care must be taken with regard to compliance by the patients themselves, and also reducing the burden on car-givers. However, in the case of a therapeutic method in which commercially available products are used together, it is necessary, for example, to administer one donepezil hydrochloride tablet once per day, and further administer one memantine hydrochloride tablet twice per day; the frequency of administration and the amounts taken are thus high, and hence problems have arisen with regard to compliance. Moreover, it is difficult to simultaneously control the release of two or more anti-dementia drugs in a single dosage form, and the current state of affairs is that specific control methods for anti-dementia drugs have not been disclosed in any publicly known literatures, and furthermore there have also been no suggestions with regard to the need to improve compliance, or techniques for producing a preparation giving a combined effect of two or more anti-dementia drugs used together.

### SUMMARY OF THE INVENTION

As described above, for the case of implementing a therapeutic method in which at least two kinds of anti-dementia drugs are used together, there is a demand for a composition which has a good therapeutic effect on dementia, and which also gives excellent compliance. More specifically, there is a demand for a composition containing at least two kinds of anti-dementia drugs, in which release of the anti-dementia drugs from the composition is controlled, whereby a combined effect of the anti-dementia drugs can be achieved as well. Furthermore, there is a demand for the development of a composition containing at least two kinds of anti-dementia drugs, in which the frequency of administration and the amount taken are reduced, and hence compliance can be improved.

In view of the above circumstances, the present inventors carried out assiduous studies in the quest for a composition which contains at least two kinds of anti-dementia drugs and which is effective for dementia, and furthermore can be administered once per day, and hence gives excellent compliance. As a result, the present inventors have discovered that the desired objects can be attained through the following construction, thus arriving at the present invention.

In other words, the present invention provides:

1. A composition comprising at least two kinds of anti-dementia drugs,

2. The composition according to item 1, further comprising at least one sustained-release portion comprising the anti-dementia drug,

3. The composition according to item 1 or 2, further comprising at least one immediate-release portion comprising the anti-dementia drug,

4. The composition according to item 2, wherein the sustained-release portion comprises at least one selected from an non-pH dependent polymeric substance and a pH dependent polymeric substance,

5. The composition according to item 3, wherein the sustained-release portion comprises at least one selected from an non-pH dependent polymeric substance and a pH dependent polymeric substance,

6. The composition according to item 1, wherein the anti-dementia drugs comprise a combination of a cholinesterase inhibitor and a compound having a mechanism of action different from that of the cholinesterase inhibitor,

7. The composition according to item 1, wherein the anti-dementia drugs comprise at least one cholinesterase inhibitor and at least one N-methyl-D-aspartate receptor antagonist,

8. The composition according to item 1, wherein the anti-dementia drugs comprise donepezil or a pharmacologically acceptable salt thereof, and memantine or a pharmacologically acceptable salt thereof,

9. The composition according to item 2, wherein the anti-dementia drug contained in the sustained-release portion is memantine hydrochloride,

10. The composition according to item 3, wherein the anti-dementia drug contained in the sustained-release portion is memantine hydrochloride, and the anti-dementia drug contained in the immediate-release portion is donepezil hydrochloride,

11. The composition according to item 2, further comprising two of the sustained-release portions, wherein one sustained-release portion comprises memantine hydrochloride as the anti-dementia drug, and another sustained-release portion comprises donepezil hydrochloride as the anti-dementia drug,

12. The composition according to item 2, wherein memantine or a pharmacologically acceptable salt thereof and donepezil or a pharmacologically acceptable salt thereof are contained in said one sustained-release portion,

13. The composition according to item 3, wherein memantine or a pharmacologically acceptable salt thereof and donepezil or a pharmacologically acceptable salt thereof are contained in said one immediate-release portion,

14. The composition according to item 4, wherein the non-pH dependent polymeric substance comprises a water-insoluble polymeric substance,

15. The composition according to item 4, wherein the pH dependent polymer comprises an enteric polymeric substance,

16. The composition according to item 5, wherein the non-pH dependent polymeric substance comprises a water-insoluble polymeric substance,

17. The composition according to item 5, wherein the pH dependent polymer comprises an enteric polymeric substance,

18. The composition according to item 2, wherein the sustained-release portion comprises a granule or a compression-molded product.

According to the composition of the present invention, not only can the effects of each of at least two kinds of anti-dementia drugs be achieved, but moreover there can be provided a novel therapeutic method due to a synergistic effect between these anti-dementia drugs. In particular, according to the present invention, there can be provided a composition containing anti-dementia drugs for which dissolution is controlled in accordance with the symptoms and state of the patient and the therapeutic method. Furthermore, according to the composition of the present invention, there can be provided a medicine that gives excellent compliance and is of excellent quality, and can be taken without anxiety by a patient exhibiting symptoms of dementia, or according to which the burden on a care-givers administering the medicine is reduced. Furthermore, according to the present invention, design of a preparation conforming to intended objectives with regard to controlling release of the anti-dementia drugs can be carried out easily without using a special manufacturing apparatus, and moreover there can be provided a simple, convenient manufacturing method for a medicinal composition in which the anti-dementia drugs are stabilized.

### DETAILED DESCRIPTION OF THE INVENTION

The following is a description of embodiments of the present invention. However, the following embodiments are merely illustrative for explaining the present invention, and it is not intended that the present invention be limited only to these embodiments. The present invention can be implemented in various modes, so long as there is no departure from the spirit and scope of the invention.

(Anti-Dementia Drug)

There are no particular limitations on an anti-dementia drug used in the present invention, so long as this drug can be used as a drug for combating dementia. The composition according to the present invention contains at least two kinds of such anti-dementia drugs. Examples of anti-dementia drugs that can be used in the present invention include, but are not limited to, cholinesterase inhibitors, NMDA receptor antagonists (e.g. memantine or the like), choline uptake enhancers (e.g. MKC-231 or the like), somatostatin release enhancers (e.g. FK960 or the like), neurotransmitter regulators (e.g. nefiracetam or the like), muscarinic M1 receptor agonists (e.g. talsaclidine or the like), benzodiazepine receptor partial inverse agonists (e.g. S-8510 or the like), and acetylcholine/noradrenaline release enhancers (e.g. T-588, T-817MA or the like) or the like. Examples of cholinesterase inhibitors include, but are limited to, tacrine, rivastigmine, galantamine, donepezil, physostigmine, pyridostigmine, neostigmine, citicoline, velnacrine, huperzine (e.g. huperzine A), metrifonate, heptastigmine, edrophonium, phenserine, tolserine, phenethylnorcymserine, ganstigmine, epastigmine, 3-[1-(phenylmethyl)piperidin-4-yl]-1-(2,3,4,5-tetrahydro-1H-1-benzazepin-8-yl)-1-propane fumarate (hereinafter referred to as “TAK-147”), 5,7-dihydro-3-[2-[1-(phenylmethyl)-4-piperidinyl]ethyl]-6H-pyrrolo[4,5-f]-1,2-benzisoxazol-6-one maleate (hereinafter referred to as “CP118954”), T-82, upreazine, and pharmacologically acceptable salts thereof. Other examples of anti-dementia drugs include, but are not limited to, vitamin E, ginkgo leaf extract, ubidecarenone, phosphatidyserine or the like. Note that the anti-dementia drug may be used either in free form, or as an organic acid salt or inorganic acid salt, with an organic acid salt or inorganic acid salt being preferable, and an inorganic acid salt being particularly preferable.

Anti-dementia drugs preferably used in the present invention are tacrine, rivastigmine, galantamine, donepezil, memantine, and pharmacologically acceptable salts thereof, and also TAK-147, and CP118954.

Particularly preferable anti-dementia drugs are tacrine, rivastigmine hydrogen tartrate, galantamine hydrobromide, donepezil hydrochloride (chemical name (±)-2[(1-benzylpiperidin-4-yl)methyl]-5,6-dimethoxyindan-1-one monohydrochloride)0, TAK-147, CP118954, and memantine hydrochloride.

There are no particular limitations on a combination of the anti-dementia drugs, which may be a combination of the anti-dementia drugs having the same mechanism of action as one another, or a combination of the anti-dementia drugs having different mechanisms of action to one another. An example is a combination of a cholinesterase inhibitor, and a compound having a mechanism of action different from that of the cholinesterase inhibitor, with a combination of the cholinesterase inhibitor and an NMDA receptor antagonist being preferable, and a combination of donepezil or a pharmacologically acceptable salt thereof, and memantine or a pharmacologically acceptable salt thereof being more preferable. A combination of donepezil hydrochloride and memantine hydrochloride is particularly preferable.

Note that the composition according to the present invention may also comprise the therapeutic drugs other than the anti-dementia drugs.

(Dose of the Drug)

There are no particular limitations on the dose of the weak basic drug or the salt thereof used in the present invention, depending on the species of the weak basic drug and aspect of the patient of each disorder, but in the case of the acetylcholinesterase inhibitor for Alzheimer-type dementia, the dose is from 0.01 to 50 mg/day. More specifically, the dose of donepezil or a pharmacologically acceptable salt thereof is from 0.01 to 50 mg/day, preferably from 0.1 to 40 mg/day, more preferably from 1 to 30 mg/day, still more preferably from 5 to 25 mg/day. The dose of rivastigmine or a pharmacologically acceptable salt thereof is from 0.01 to 50 mg/day, preferably from 0.1 to 30 mg/day, more preferably from 1 to 20 mg/day, still more preferably from 1 to 15 mg/day. The dose of galantamine or a pharmacologically acceptable salt thereof is from 0.01 to 50 mg/day, preferably from 0.1 to 40 mg/day, more preferably from 1 to 30 mg/day, still more preferably from 2 to 25 mg/day.

Moreover, in the case of memantine or a pharmacologically acceptable salt thereof which acts as a NMDA receptor antagonist, the dose is from 0.5 to 100 mg/day, preferably from 1 to 100 mg/day, more preferably from 1 to 40 mg/day, still more preferably from 5 to 25 mg/day.

The anti-dementia drug dose can be divided with the composition being administered a plurality of times per day, but the composition is preferably administered at least once per day. The composition according to the present invention thus preferably comprises at least one day's dose of each of at least two kinds of the anti-dementia drugs.

(Control of Release)

The composition according to the present invention enables controlled release from the composition containing at least two kinds of the anti-dementia drugs to be attained easily; for example, a method and form of administration in which administration is carried out once per day or less frequently than this can be realized. The term “controlled release” used in the present invention means that the release of the drugs from the composition is controlled in accordance with the object. When realizing such controlled release in the present invention, the release of at least two kinds of the anti-dementia drugs can be controlled from a single preparation through either a sustained-release capability or immediate-release function alone, or a combination thereof. The term “sustained-release” herein not only indicates the anti-dementia drug being released more gradually over time than with immediate-release, but also includes extended-release or pulsed-release in which release of the drug starts after a certain period of time, and prolong action in which the drug concentration is maintained over time. Moreover, with “immediate-release”, the objective is for the drug to be released rapidly after administration, for example for it to be possible to release at least 85% of the anti-dementia drug within 1 to 3 hours after commencement of dissolution in a dissolution test.

With the composition according to the present invention, there are no particular limitations on the combination of the types and amounts of the anti-dementia drugs, or the types of controlled release. For example, in the case that there are two anti-dementia drugs, one anti-dementia drug may be made to be immediate-release, and the other sustained-release. That is, two anti-dementia drugs that have hitherto been administered with different frequencies, for example an anti-dementia drug hitherto administered twice per day and an anti-dementia drug hitherto administered once per day, can be combined into a composition form that is administered once per day. An example is a composition containing at least two kinds of the anti-dementia drugs obtained by making an effective dose of an anti-dementia drug that is usually administered twice per day such as tacrine, memantine, galantamine or rivastigmine be sustained-release, and further adding donepezil, which is usually administered once per day.

As another example, one anti-dementia drug can be controlled to be immediate-release and sustained-release, while the other is made to be sustained-release or immediate-release. An example is a composition in which 10 mg of memantine hydrochloride and 10 mg of donepezil hydrochloride are made to be immediate-release, and another 10 mg of memantine hydrochloride is controlled so as to be released 6 to 8 hours after administration.

As yet another example, two anti-dementia drugs that are usually used in different dose regimen can both be made to be sustained-release, or both be made to be immediate-release. In this case, the methods of making the anti-dementia drugs be sustained-release or immediate-release may be of the same type of control of release, or a different type. For example, for a single composition, control can be carried out such that both 10 mg of donepezil hydrochloride and 20 mg of memantine hydrochloride are released gradually 6 to 12 hours after administration. Alternatively, the control of release of the two drugs can be carried out such that the donepezil hydrochloride is released gradually 6 to 12 hours after administration, and the memantine hydrochloride is subjected to pulsed-release immediately after administration and 6 to 8 hours after administration.

Yet another example is a composition in which two anti-dementia drugs are both controlled to be immediate-release. An example is a composition containing 10 mg of memantine hydrochloride and 10 mg of donepezil hydrochloride, this being a composition enabling good anti-dementia effects to be achieved upon administration only once per day and with a reduced dose of the drugs as compared to the case of administering a commercially available preparation of 10 mg of memantine hydrochloride twice per day and a commercially available preparation of 10 mg of donepezil hydrochloride once per day. Yet another example of a composition according to the present invention is a composition comprising an immediate-release portion, which may be a composition containing 10 mg of memantine hydrochloride and 5 mg of donepezil hydrochloride that is administered twice per day. Note that in the case of making the composition according to the present invention contain memantine hydrochloride and donepezil hydrochloride, there are no particular limitations on the amounts of the memantine hydrochloride and the donepezil hydrochloride.

There are no particular limitations on the anti-dementia drug contained in the composition according to the present invention, but from the standpoint of controlling release, it is effective for the basic drugs or the salts thereof which are less soluble in an alkaline aqueous solution than in an acidic aqueous solutions and the solubility of the basic drugs or the salts thereof for a pH of an aqueous solution changes near the neutral pH. Moreover, according to the composition of the present invention, control can be carried out simultaneously for the anti-dementia drug for which the change in solubility at pH in an aqueous solution around a neutral pH is relatively small, and the anti-dementia drug for which this change is relatively large. The anti-dementia drug used in the present invention is, for example, a basic drug or a salt thereof for which the pKa of a basic functional group of the anti-dementia is from 7 to 12, preferably from 7.5 to 11, more preferably from 8 to 10.5, most preferably from 8.5 to 10.5. For example, donepezil hydrochloride is a basic drug with pKa=8.90, and memantine hydrochloride is a basic drug with pKa=10.27.

(Embodiment of Composition)

The composition according to the present invention comprises at least one sustained-release portion for performing sustained-release function when controlling the release of at least two kinds of anti-dementia drugs. The composition further comprises at least one immediate-release portion for performing immediate-release function. Here, the term “containing at least one immediate-release portion or sustained-release portion” means that there may be one immediate-release portion or sustained-release portion, or a plurality of immediate-release portions or sustained-release portions, in the composition. A composition containing a sustained-release portion containing at least one anti-dementia drug is preferable. Also preferable is a composition containing an immediate-release portion containing at least one anti-dementia drug. More preferable is a composition containing a sustained-release portion containing at least one anti-dementia drug, and an immediate-release portion containing at least one of other anti-dementia drug. Here, the sustained-release portion in the present invention has a sustained-release function for at least one of the anti-dementia drugs. In this case, the form of the composition may be such that one sustained-release portion constitutes the whole composition, or may be such that the composition has at least one sustained-release portion as a part of the composition. Examples of the former include tablets or granules having a sustained-release film coating, and a matrix type sustained-release preparation having a wax or a resin as a base material. Moreover, examples of the latter include tablets formed from a mixture of sustained-release granules constituting a sustained-release portion and immediate-release granules constituting an immediate-release portion, a capsule preparation obtained by filling a capsule with sustained-release granules and immediate-release granules, and press-coated tablets in which an outer layer constituting an immediate-release portion is formed on an inner core constituting a sustained-release portion. Moreover, the composition may be of a type in which a tablet containing sustained-release granules constituting a sustained-release portion is further coated with a sustained-release film so as to give the composition as a whole a sustained-release function. There is, however, no limitation to the above embodiments. Moreover, there are no particular limitations on the blending state of each anti-dementia drug in the composition or in an immediate-release portion or a sustained-release portion; the anti-dementia drug may be dispersed uniformly in the composition, or may be contained in only one part of the composition, or may be contained such that there is a concentration gradient.

Specific embodiments of the composition according to the present invention are given below, but there is no limitation thereto. Here, examples are given of various types of composition that can be administered once per day and contain, as the anti-dementia drugs, donepezil hydrochloride, which is usually administered once per day, and memantine hydrochloride, which is usually administered twice per day.

(Matrix Type Preparation)

A first example is a matrix type preparation. An aqueous solution of hydroxypropyl cellulose (HPC-L; Nippon Soda Co., Ltd) is added to a mixture of donepezil hydrochloride (manufactured by Eisai Co. Ltd.), memantine hydrochloride (manufactured by Lachema s.r.o. Czech Republic), ethylcellulose (Ethocel 10FP, manufactured by Dow Chemical Company, USA and the like), Eudragit L100-55 (manufactured by Röhm GmbH & Co. KG, Darmstadt, Germany), and lactose, and wet granulation is carried out, and then the resulting granules are heat dried using a tray dryer, and then sieved to obtain the desired granule size. After sizing, magnesium stearate (Mallinckrodt Baker, Inc. USA) is added to the resulting sustained-release granules and mixing is carried out, and then a rotary tabletting machine is used to form a tablet, whereby this tablet containing 10 mg of donepezil hydrochloride and 20 mg of memantine hydrochloride can be obtained. Alternatively, it is also possible to prepare sustained-release granules for each of memantine hydrochloride and donepezil hydrochloride, then add sodium stearyl fumarate and carry out mixing, and then use a rotary tabletting machine to obtain a tablet. In this case, for each of the types of sustained-release granules, the amount the non-pH dependent polymeric substance or the pH dependent polymeric substance according to the present invention can be varied in accordance with the release profile of two drugs. In any case, both donepezil hydrochloride and memantine hydrochloride can be made to be sustained-release, and hence such tablet can be used as a tablet form administerable once per day.

(Gel Matrix Type Preparation)

A second example is a gel matrix type preparation. Donepezil hydrochloride (manufactured by Eisai Co. Ltd.), memantine hydrochloride (manufactured by Lachema s.r.o. Czech Republic), and polyethylene oxide (Polyox, manufactured by Dow Chemical Company, USA), carboxyvinyl polymer (manufactured by BF Goodrich), and hydroxypropyl cellulose (HPC-L; Nippon Soda Co., Ltd, Japan), each of the above three polymers be water-swellable or forms a gel in water, are mixed together, and compression molding is carried out using a rotary tabletting machine, whereby a compression-molded product can be obtained as a sustained-release portion. A film-coated tablet can then be obtained by using Opadry Yellow (Japan Colorcon) to further coat with a water-soluble film coating (coating amount: 5 mg/tablet) containing hydroxypropyl methylcellulose as a main component thereof. According to the resulting tablet, both donepezil hydrochloride and memantine hydrochloride can be made to be sustained-release, and hence the tablet can be used as a tablet form administerable once per day.

(Multi-Layered Tablet)

A third example is a tablet in which a plurality of layers are stacked. These layers may be a combination of sustained-release portions and immediate-release portions having different functions as appropriate based on the release profiles of the anti-dementia drugs. An example is a two-layer tablet in which the first layer constituting an immediate-release portion contains donepezil hydrochloride, and the second layer constituting a sustained-release portion contains memantine hydrochloride. In this case, Eudragit RS (manufactured by Röhm GmbH & Co. KG, Darmstadt, Germany) and Eudragit L100-55 (manufactured by Röhm GmbH & Co. KG, Darmstadt, Germany) are contained in the second layer. Moreover, a sustained release function may be conferred using polyethylene oxide and carboxyvinyl polymer (manufactured by BF Goodrich) as for the gel matrix type preparation. Through such a construction, release of memantine hydrochloride from the second layer can be made to be sustained while making release of donepezil hydrochloride from the first layer be quick. Moreover, in such two-layer tablet, the drugs in the two layers can be replaced, i.e. such that memantine hydrochloride is released quickly from the first layer, and donepezil hydrochloride is released in a sustained way from the second layer. Alternatively, the composition may be of a form administerable once per day in which the first layer is made to be a sustained-release portion containing 10 mg of donepezil hydrochloride and 10 mg of memantine hydrochloride, and the second layer is made to be a sustained-release portion from which 10 mg of memantine hydrochloride is released in a pulsed way. Another example is a composition that is a two-layer tablet, with both layers being made to be an immediate-release portion, and donepezil hydrochloride and memantine hydrochloride being contained in the respective layers. In this case, the two immediate-release portions may have the same immediate-release functions as each other, or different immediate-release functions, the control of release being carried out freely in accordance with the types of the anti-dementia drugs and so on.

(Press-Coated Tablet)

A fourth example is a press-coated tablet having an inner core layer, and an outer layer covering the inner core layer. Examples are as follows: (1) A dried-coated tablet containing donepezil hydrochloride in the outer layer, which is an immediate-release portion, and memantine hydrochloride in the inner core layer, which is a sustained-release portion. In this case, the inner core layer may contain ethylcellulose (Ethocel 10FP, manufactured by Dow Chemical Company, USA) and Eudragit L100-55 (manufactured by Röhm GmbH & Co. KG, Darmstadt, Germany), so that release of the donepezil hydrochloride from the outer layer can be made to be quick, and release of the memantine hydrochloride from the inner core layer can be made to be sustained. (2) A composition in which both donepezil hydrochloride and memantine hydrochloride are released quickly from an outer layer containing both of the drugs, and then after a certain period of time has elapsed, memantine hydrochloride is released in a pulsed way from an inner core layer. To make the release pulsed, the inner core layer can be surrounded by a coating layer for pulsed-release, or a disintegrant can be contained in the inner core layer. (3) A composition having as an inner core layer a two-layer tablet comprising an immediate-release portion from which memantine hydrochloride is released quickly, and a sustained-release portion from which memantine hydrochloride is released in a sustained way, and an outer layer containing donepezil hydrochloride.

(Multi-Granule Preparation)

A fifth example is a composition containing a plurality of types of granules. Each of the types of granules can be made to be immediate-release, sustained-release, pulsed-release or the like, so as to freely establish the desired dissolution profile. For example, (1) immediate-release granules containing memantine hydrochloride, sustained-release granules containing donepezil hydrochloride, and pulsed-release granules containing memantine hydrochloride can be contained in the composition, whereby upon one administration, the interval between the times when the blood plasma concentration of memantine hydrochloride reaches a maximum is 8 hours or more, and the donepezil hydrochloride is released gradually after administration. Alternatively, (2) a preparation form administerable once per day can be produced by making sustained-release granules for which dissolution commences 2 hours, 4 hours, 6 hours, or 8 hours after dissolution contain 5 mg of memantine hydrochloride, and combining these with granules from which 10 mg of donepezil hydrochloride is released in a sustained way. There is no limitation to the above dissolution profiles. Moreover, there are also no limitations on the dosage form of the preparation, which may be a granular preparation obtained by mixing the various types of granules together, or alternatively a tablet obtained by compression molding the various types of granules, or a capsule preparation obtained by filling the various types of granules into HPMC capsules or the like.

(Multi-Layered Granules)

A sixth example is granules in which layers containing anti-dementia drugs are multi-layered on core particles of nonpareil or the like. An example is granules in which a plurality of layers containing the anti-dementia drugs are multi-layered on Nonpareil 101 (Freund Corporation, Japan) by alternately coating with a film coating liquid containing memantine hydrochloride and a film coating liquid containing donepezil hydrochloride. In this case, release of the anti-dementia drugs may be controlled by changing the concentration of the anti-dementia drug in each layer. Alternatively, sustained-release granules may be formed in which thin layers containing ethylcellulose and a plasticizer are provided between the layers containing the drugs, and as an outermost layer. Alternatively, a sustained-release function can be conferred to each of the layers containing the drugs by mixing the anti-dementia drugs with ethylcellulose, Eudragit RS or the like in advance. Note that such granules can also be obtained by using granules containing at least one anti-dementia drug as the core particles instead of Nonpareil, and multilayers containing the same anti-dementia drug or a different anti-dementia drug on these core particles. The resulting granules may be used alone, or a plurality of types of such granules may be combined; the granules may be used as the composition according to the present invention either as a granular preparation as is, or as a capsule preparation filled into HPMC capsules.

(Film-Coated Tablet)

A seventh example is a film-coated tablet. Memantine hydrochloride, donepezil hydrochloride, crystalline cellulose, lactose, and corn starch are mixed together, an aqueous solution of hydroxypropyl cellulose is added thereto, and wet granulation is carried out, and then the resulting granules are heat dried using a tray dryer, and then sieved to obtain the desired granule size. After sizing, magnesium stearate is added to the resulting immediate-release granules and mixing is carried out, and then a rotary tabletting machine is used to form a tablet, whereby a compression-molded product that is an immediate-release portion containing donepezil hydrochloride and memantine hydrochloride is obtained. Immediate-release film-coated tablets can then be obtained by using Opadry Yellow (Japan Colorcon) to further coat with a water-soluble film coating having hydroxypropyl methylcellulose as a main component thereof. Alternatively, instead of a water-soluble film coating, coating may be carried out with a mixture of a water-insoluble polymer such as ethylcellulose or Eudragit RS, and a water-soluble polymer or a plasticizer, so as to obtain sustained-release film-coated tablet. Moreover, taking the compression-molded product that constitutes the immediate-release portion as mini-tablets, a plurality of film-coated tablets having different thicknesses or compositions of the sustained-release film may be prepared, and then filled into HPMC capsules.

An eighth example is a composition in which a compression-molded product is taken as an immediate-release portion, and sustained-release granules are dispersed in this compression-molded product. An example is a tablet obtained by mixing memantine hydrochloride and ethylcellulose together, and granulating to prepare sustained-release granules, and then mixing these sustained-release granules with donepezil hydrochloride, a diluent, a binder and so on, and compression-molding this mixture. The sustained-release granules may be granules having a single dissolution profile, or granules having a plurality of dissolution profiles as in the fifth example, or stacked granules as in the seventh example. Moreover, as the sustained-release portion, instead of sustained-release granules, a liposome or micro-capsules containing an anti-dementia drug may be contained.

(Dosage Form)

There are no particular limitations on the dosage form of the composition according to the present invention, which may be any dosage form including tablets, capsules, granules, fine granules, powders, orally rapid disintegrating tablets, ointments, injections, poultices, liquids, preparations for per-tube administration, inhalants, jellies or the like. The dosage form is preferably one suitable for oral administration such as tablets, capsules, granules, fine granules, orally rapid disintegrating tablets, liquids, preparations for per-tube administration, or jellies, with tablets, capsules, granules, fine granules, or orally rapid disintegrating tablets, being particularly preferable.

(Additives for Controlling Release)

A sustained-release portion in the composition according to the present invention comprises at least one non-pH dependent polymeric substance or pH dependent polymeric substance for controlling release of the anti-dementia drug, and preferably comprises the non-pH dependent polymeric substance and the pH dependent polymeric substance.

(Non-pH Dependent Polymeric Substance)

The non-pH dependent polymeric substance used in the present invention is a polymeric substance whose charge state hardly changes under pH conditions generally found in the gastrointestinal tract, specifically from pH 1 to pH 8. This means, for example, a polymeric substance that does not have functional groups whose charge state changes depending on the pH such as basic functional groups such as amino groups or acidic functional groups such as carboxylic acid groups. Note that in the present invention, the non-pH dependent polymeric substance can be included for giving the composition according to the present invention a sustained-release function, but may also be included for another purpose. Moreover, the non-pH dependent polymeric substance used in the present invention may be water-insoluble, or may swell in water or dissolve in water to form a gel. Examples of the water-insoluble non-pH dependent polymeric substance include, but are not limited to, cellulose ethers, cellulose esters, and methacrylic acid-acrylic acid copolymers (trade name Eudragit, manufactured by Röhm GmbH & Co. KG, Darmstadt, Germany). Examples include, but are not limited to, cellulose alkyl ethers such as ethylcellulose (trade name Ethocel, manufactured by Dow Chemical Company, USA), ethyl methylcellulose, ethyl propylcellulose or isopropylcellulose, butylcellulose or the like, cellulose aralkyl ethers such as benzyl cellulose, cellulose cyanoalkyl ethers such as cyanoethylcellulose or the like, cellulose organic acid esters such as cellulose acetate butyrate, cellulose acetate, cellulose propionate or cellulose butyrate, cellulose acetate propionate or the like, ethyl acrylate-methyl methacrylate copolymers (trade name Eudragit NE, manufactured by Röhm GmbH & Co. KG, Darmstadt, Germany), aminoalkyl methacrylate copolymers RS or the like (trade names Eudragit RL, Eudragit RS). There are no particular limitations on the mean particle diameter of the water-insoluble polymer used in the present invention, but usually the lower this mean particle diameter the better performance, with the mean particle diameter preferably being from 0.1 to 100 μm, more preferably from 1 to 50 μm, particularly preferably from 3 to 15 μm, most preferably from 5 to 15 μm. Moreover, examples of the water-soluble or water-swelling non-pH dependent polymeric substance include, but are not limited to, polyethylene oxide (trade name Polyox, manufactured by Dow Chemical, molecular weight 100,000 to 7,000,000), low-substituted hydroxypropyl cellulose (trade name L-HPC, manufactured by Shin-Etsu Chemical, Japan), hydroxypropyl cellulose (trade name HPC, manufactured by Nippon Soda, Co., Ltd, Japan), hydroxypropyl methylcellulose (trade names METOLOSE 60SH, 65SH, 90SH, manufactured by Shin-Etsu Chemical, Japan), and methylcellulose (trade name METOLOSE SM, manufactured by Shin-Etsu Chemical, Japan).

Note that in the present invention, a single non-pH dependent polymeric substance may be contained in the composition, or a plurality of the non-pH dependent polymeric substances may be contained. The non-pH dependent polymeric substance used in the present invention is preferably a water-insoluble polymeric substance, preferably ethylcellulose, an ethyl acrylate-methyl methacrylate copolymer (trade name Eudragit NE), or an aminoalkyl methacrylate copolymer (trade name Eudragit RL, Eudragit RS). It is particularly preferable that non-pH dependent polymeric substance be at least one of ethylcellulose and the aminoalkyl methacrylate copolymer. It is most preferable that non-pH dependent polymeric substance be ethylcellulose. There are no particular limitations on the amount of the at least one non-pH dependent polymeric substance contained in the composition; this amount can be adjusted as appropriate in accordance with the purpose such as controlling sustained-release of the drug.

(pH Dependent Polymeric Substance)

The pH dependent polymeric substance used in the present invention is a polymer whose charge state changes under pH conditions generally found in the gastrointestinal tract, specifically from pH 1 to pH 8. This means, for example, a polymeric substance having functional groups whose charge state changes depending on the pH such as basic functional groups such as amino groups or acidic functional groups such as carboxylic acid groups. The pH dependent functional groups of the pH dependent polymeric substance are preferably acidic functional groups, with the pH dependent polymeric substance most preferably having carboxylic acid groups.

The pH dependent polymeric substance used in the present invention may be water-insoluble, or may swell in water or dissolve in water to form a gel. Examples of the pH dependent polymeric substances used in the present invention include, but are not limited to, enteric polymeric substances. Examples of the enteric polymeric substances include, but are not limited to, methacrylic acid-methyl methacrylate copolymers (Eudragit L100, Eudragit S100, manufactured by Röhm GmbH & Co. KG, Darmstadt, Germany), methacrylic acid-ethyl acrylate copolymers (Eudragit L100-55, Eudragit L30D-55, manufactured by Röhm GmbH & Co. KG, Darmstadt, Germany), hydroxypropyl methylcellulose phthalate (HP-55, HP-50, manufactured by Shin-Etsu Chemical, Japan), hydroxypropyl methylcellulose acetate succinate (AQOAT, manufactured by Shin-Etsu Chemical, Japan), carboxymethyl ethylcellulose (CMEC, manufactured by Freund Corporation, Japan), cellulose acetate phthalate or the like. Examples of the pH dependent polymeric substances that swell in water or dissolve in water to form a gel include, but are not limited to, alginic acid, pectin, carboxyvinyl polymer, carboxymethyl cellulose or the like. In the present invention, a single pH-dependent polymeric substance may be contained in the composition, or a plurality of pH-dependent polymeric substances may be contained. The pH dependent polymeric substance used in the present invention is preferably an enteric polymeric substance, more preferably a methacrylic acid-ethyl acrylate copolymer, a methacrylic acid-methyl methacrylate copolymer, hydroxypropyl methylcellulose phthalate, or hydroxypropyl methylcellulose acetate succinate, particularly preferably a methacrylic acid-ethyl acrylate copolymer.

When using the pH dependent polymeric substance in the manufacturing process of the composition according to the present invention, commercially available products of powder types or granular types, or suspensions type in which the pH dependent polymeric substance has been dispersed in a solvent in advance can be used as is, or such commercially available products can be used by dispersing them in water or an organic solvent. The lower the particle diameter of the pH dependent polymeric substance the better performance, with the pH dependent polymeric substance preferably being of the powder types. In the case of a methacrylic acid-ethyl acrylate copolymer, an example is Eudragit L100-55. There are no particular limitations on the mean particle diameter of the pH dependent polymeric substance used in the present invention, but the mean particle diameter is preferably from 0.05 to 100 μm, more preferably from 0.05 to 70 μm, most preferably from 0.05 to 50 μm. Moreover, there are no particular limitations on the amount of the pH dependent polymeric substance, for example, in the case of the enteric polymeric substance, the amount is generally from 0.1 to 90 parts by weight, preferably from 1 to 70 parts by weight, more preferably from 5 to 60 parts by weight, particularly preferably 10 to 50 parts by weight, based on 100 parts by weight of the composition.

(Additives)

The composition according to the present invention may further comprise any of various additives, such as any of various pharmacologically acceptable carriers such as diluents, lubricants, binders and disintegrants, as well as preservatives, colorants, sweeteners, plasticizers, film coating agents and so on as necessary. Examples of the diluents include, but are not limited to, lactose, mannitol, dibasic calcium phosphate, starch, pregelatinized starch, crystalline cellulose (Asahi Kasei Corporation, Japan), light silicic anhydride, synthetic aluminum silicate, magnesium aluminate metasilicate or the like. Examples of the lubricants include, but are not limited to, magnesium stearate (Mallinckrodt Baker, Inc. USA), calcium stearate, talc, sodium stearyl fumarate or the like. Examples of the binders include, but are not limited to, hydroxypropyl cellulose, methylcellulose, sodium carboxymethyl cellulose, hydroxypropyl methylcellulose, polyvinylpyrrolidone or the like. Examples of the disintegrants include, but are not limited to, carboxymethyl cellulose, calcium carboxymethyl cellulose, croscarmellose sodium, sodium carboxymethyl starch, low-substituted hydroxypropyl cellulose or the like. Examples of the preservatives include, but are not limited to, paraoxybenzoic acid esters, chlorobutanol, benzyl alcohol, phenethyl alcohol, dehydroacetic acid, sorbic acid or the like. Preferable examples of colorants include, but are not limited to, water-insoluble lake pigments, natural pigments (e.g. β-carotene, chlorophyll, red ferric oxide), yellow ferric oxide, red ferric oxide, and black iron oxide. Preferable examples of the sweeteners include, but are not limited to, sodium saccharin, dipotassium glycyrrhizate, aspartame, stevia or the like. Examples of the plasticizers include, but are not limited to, glycerol fatty acid esters, triethyl citrate, propylene glycol, polyethylene glycol or the like. Examples of the film coating bases include, but are not limited to, hydroxypropyl methylcellulose, hydroxypropyl cellulose or the like.

(Manufacturing Methods)

To manufacture the composition according to the present invention, a single conventional method, or a combination of the conventional methods, can be used. For example, when manufacturing granules containing the anti-dementia drugs as a sustained-release portion or an immediate-release portion in the present invention, granulation method is the main operation, but this may be combined with other operations such as mixing, drying, sieving, and classification. As the granulation method, for example, a wet granulation method in which the binder and a solvent are added to the powder and granulation is carried out, a dry granulation method in which the powder is compressed and granulation is carried out, a molten granulation method in which the binder that melts on heating is added and heated, and granulation is carried out or the like can be used. Furthermore, in accordance with the granulation method, an operating method such as mixing granulation method using a planetary mixer, a screw mixer or the like, high-speed mixing granulation method using a Henschel mixer, a Super mixer or the like, extruding granulation method using a cylindrical granulator, a rotary granulator, a screw extruding granulator, a pellet mill type granulator or the like, wet high-shear granulation method, fluidized-bed granulation method, compression granulation method, crushing granulation method, or spraying granulation method can be used. After the granulation, drying using a dryer, a fluidized bed or the like, cracking, and sieving can be carried out to obtain the granules or fine granules for use. Moreover, a granulation solvent may be used when preparing the composition according to the present invention. There are no particular limitations on such a granulation solvent, which may be water or any of various organic solvents, for example, water, a lower alcohol such as methanol or ethanol, a ketone such as acetone or methyl ethyl ketone, methylene chloride, or a mixture thereof.

(Method of Manufacturing Granules)

For sustained-release granules contained in the composition according to the present invention, at least one anti-dementia drug and at least one selected from the non-pH dependent polymeric substances and the pH dependent polymeric substances are mixed together, the diluent and the binder are added as necessary, and granulation is carried out to obtain granular matters. The resulting granular matters is dried using a tray dryer, a fluidized bed dryer or the like, and sieving is carried out using a mill or an oscillator, whereby the sustained-release granules can be obtained. Alternatively, as a method of manufacturing the sustained-release granules in the present invention, it is possible to add at least one anti-dementia drug, at least one selected from the non-pH dependent polymeric substances and the pH dependent polymeric substances, and as necessary a diluent and the binder by using a dry compactor such as a roller compactor or a slug tabletting machine, and to carry out compression molding while mixing, and then carry out granulation by cracking down to a suitable size. Granular matters prepared using such a granulator may be used as is as granules or fine granules according to the present invention, or may be further cracked using a power mill, a roll granulator, a rotor speed mill or the like, and are sieved to obtain the sustained-release granules. Note that the immediate-release granules can also be manufactured as for the sustained-release granules.

(Method of Manufacturing Compression-Molded Product)

A compression-molded product can be manufactured as a sustained-release portion or an immediate-release portion which contains the anti-dementia drug, or as the composition according to the present invention using a single conventional method, or a combination of the conventional methods. For example, at least one anti-dementia drug, at least one selected from the non-pH dependent polymeric substances and the pH dependent polymeric substances, the diluents such as mannitol or lactose, the binders such as polyvinylpyrrolidone or crystalline cellulose, the disintegrants such as carmellose sodium or crospovidone, and the lubricants such as magnesium stearate or talc are used, and tabletting is carried out using an ordinary method, whereby the compression-molded product can be obtained. In this case, tabletting is the main operation in the method of manufacturing the compression-molded product, but this may be combined with other operations such as mixing, drying, sugar coating formation, and coating. Examples of the method for tabletting include, but are not limited to, direct compression-molding in which at least one anti-dementia drug and pharmacologically acceptable additives are mixed together and then the mixture is directly compression molded into tablets using a tabletting machine, and dry granule compression and wet granule compression in which sustained-release granules or immediate-release granules according to the present invention are subjected to compression molding after adding the lubricants or the disintegrants as necessary. There are no particular limitations on the tabletting machine used in the compression-molding; for example, a single-punch tabletting machine, a rotary tabletting machine, or a press-coated tabletting machine can be used.

(Coating Method)

The sustained-release granules or the immediate-release granules, or the compression-molded products containing the anti-dementia drug of the present invention can be used as is in the form of granules or tablets as the composition of the present invention, but may also be subjected to further processing to manufacture the composition. For example, the compression-molded products or granules can be given a film coating using a film base material such as ethylcellulose, casein, methylcellulose, hydroxypropyl methylcellulose, methacrylic acid copolymer L, cellulose acetate phthalate, shellac or the like, or given a sugar coating using a sugar coating liquid containing saccharose, sugar alcohol, gum arabic powder, talc or the like, thus producing film-coated tablets or sugar-coated tablets. A preferable solvent in this coating technique is purified water, but an organic solvent such as alcohols, ketones, ethers or chlorinated hydrocarbons, or a mixture thereof can also be used. For example, ethanol, acetone, methylene chloride or the like can be used as an organic solvent. Moreover, as the coating apparatus, the apparatus ordinarily used in coating techniques for manufacturing medicines can be used, with examples including a spray coating apparatus in which the coating is carried out by spraying a coating liquid or the like, and a rotor fluidized bed granulator for layering.

(Other Manufacturing Methods)

In the case of manufacturing capsule preparations, the capsule preparations can be manufactured by filling the sustained-release granules, the immediate-release granules, or mini-tablets into hard gelatin capsules or HPMC capsules using an automatic capsule filling machine.

Alternatively, in the case of the preparations administerable through a tube or a dry syrup that is used mixed with water or the like when taken, the sustained-release granules or the immediate-release granules can be mixed with thickeners or dispersants so as to disperse these granules, the mixture then being made into granules or tablets. Furthermore, liquids or jellies can be made using water, dispersants, emulsifiers, thickeners, preservatives, pH adjustors, sweeteners, flavorings, fragrances and so on. However, with respect to other manufacturing methods, there are no limitations to the above.

(Dissolution Test)

With the composition of the present invention, release of the anti-dementia drugs can be controlled. A dissolution test method described in the Japanese Pharmacopoeia 14^{th} Edition, USP or the like can be used for identifying a means of controlled release of the anti-dementia drugs, or for evaluating the control sate of release. For example, measurement can be carried out using the first dissolution test method (rotating basket method), the second dissolution test method (paddle method), or the third dissolution test method (flow-through cell method) in the Japanese Pharmacopoeia. The composition with specified dissolution profile according to the present invention can be obtained using such a test method. For example, according to the present invention, the composition can be obtained in which, for each of two anti-dementia drugs contained in the composition, in a dissolution test, dissolution with little pH dependence can be secured during an early stage of dissolution, and then in a late stage of dissolution, the ratio of dissolution ratio in an acidic solution to dissolution ratio in a neutral solution decreases over time as the dissolution test proceeds.

(Controlled-Release Composition)

The composition according to the present invention comprises at least two kinds of the anti-dementia drugs, with it being possible to control the dissolution of these anti-dementia drugs together or individually.

For example, the present invention provides a composition, upon carrying out measurement with a paddle rate of 50 rpm using the second dissolution test method in the Japanese Pharmacopoeia, in which at least one anti-dementia drug contained in the composition has the dissolution ratio in a 0.1 N hydrochloric acid solution being from 20 to 50% at a dissolution time of 1 hour, and being from 85 to 100% at a dissolution time of 3 hours. Moreover, the composition may be such that, under the same dissolution conditions, in which at least one anti-dementia drug contained in the composition has the dissolution ratio in a 0.1 N hydrochloric acid solution being from 5 to 20% at a dissolution time of 1 hour, and being from 90 to 100% at a dissolution time of 8 hours. By combining such dissolution characteristics, the anti-dementia drugs contained in the composition according to the present invention can thus all be made to be sustained-release or immediate-release. Alternatively, one of the anti-dementia drugs can be made to be immediate-release, and the remainder sustained-release.

Technologies for making the release be sustained can be used, in particular, to solve the problems of compliance for the patient taking the anti-dementia drugs.

For example, the present invention provides a composition, upon carrying out measurement with a paddle rate of 50 rpm using the second dissolution test method in the Japanese Pharmacopoeia, in which at least one anti-dementia drug contained in the composition has the ratio of dissolution ratio in the 0.1 N hydrochloric acid solution to the dissolution ratio in a 50 mM phosphate buffer, pH 6.8 at a dissolution time of 3 hours being from 0.3 to 1.3. That is, the dissolution ratio while residing in the stomach is suppressed, and the speed of dissolution is made low, whereby the drug concentration in the blood plasma can be prevented from rising suddenly. The onset of the side effects can thus be prevented, and there is a contribution to making drug release-sustained.

In another example, the present invention provides a composition, upon carrying out measurement with a paddle rate of 50 rpm using the second dissolution test method in the Japanese Pharmacopoeia, in which at least one anti-dementia drug contained in the composition has the ratio of the dissolution ratio in a 0.1 N hydrochloric acid solution to the dissolution ratio in a 50 mM phosphate buffer, pH 6.8 decreases with dissolution time up to the dissolution time at which the dissolution ratio in a 50 mM phosphate buffer, pH 6.8 is 90%. That is, the dissolution ratio in the stomach is kept low, and furthermore a decrease in the drug bioavailability as the composition passes from the stomach into the small intestine can be inhibited, and hence the pharmacological effects can be achieved reliably.

The composition according to the present invention can control the release of a plurality of the anti-dementia drugs therein simultaneously. For example, in the case of the composition containing two drugs having different solubilities to one another at pH 6.8, by producing the two-layered tablets formed from the first layer containing the less soluble anti-dementia drug and the second layer containing the more soluble anti-dementia drug, and making the total amount of the non-pH dependent polymeric substances and the pH-dependent polymeric substances added as the release-controlling substances be higher in the second layer than in the first layer, a desired sustained-release preparation can be obtained.

As another example, in the case of the composition containing two drugs having different solubility ratios between in a 0.1 N hydrochloric acid solution and in a buffer of pH 6.8 (i.e. solubility in the 0.1 N hydrochloric acid solution/solubility in the buffer of pH 6.8), by producing the two-layered tablet formed from the first layer containing the anti-dementia drug having the lower solubility ratio and the second layer containing the anti-dementia drug having the higher solubility ratio, and making the amount of the pH dependent polymeric substances based on 1 part by weight of the non-pH dependent polymeric substances be higher in the second layer than in the first layer, a desired sustained-release preparation can be obtained. Furthermore, as still another example, for two drugs having both (1) a different solubility at pH 6.8 and (2) a different solubility ratio between in a 0.1 N hydrochloric acid solution and in a buffer of pH 6.8, by suitably adjusting both the total amount of the non-pH dependent polymeric substances and the pH dependent polymeric substances added as the release-controlling substances, and the amount of the pH dependent polymeric substances relative to the non-pH dependent polymeric substances as in the above examples, a desired sustained-release preparation can be obtained.

Alternatively, it can be expected in the still another example that a synergistic effect can be obtained for the composition in which the dissolution profile of the drug in the first layer containing the anti-dementia drug is about the same as the dissolution profile of the drug in the second layer. More specifically, in the case of the composition in which two or more of the anti-dementia drug is contained in the immediate-release portion, when carrying out the dissolution test with a paddle rate of 50 rpm using the first dissolution test solution (pH 1.2, temperature of test solution being 37±0.5° C.) according to the Japanese Pharmacopoeia, a ratio of a dissolution rate of one anti-dementia drug in the composition to a dissolution rate of other anti-dementia drug is from 0.85 to 1.15 at a dissolution time of 2 hour.

Moreover, in the case of the composition in which two or more of anti-dementia drug is contained in the sustained-release portion, when carrying out the dissolution test using 50 mM phosphate buffer, pH 6.8 according to the second dissolution test method of the Japanese Pharmacopoeia, a ratio of a dissolution rate of one anti-dementia drug in the composition to a dissolution rate of the other anti-dementia drug is from 0.85 to 1.15 at a specified dissolution time from 3 hours to 8 hours (for examples, dissolution times of 4 hours, 6 hours, 8 hours or the like).

In the present invention, regardless of whether the composition comprises one sustained-release portion or a plurality of sustained-release portions, the content of release-controlling substances (the non-pH dependent polymeric substances and the pH dependent polymeric substances) in the sustained release portion is generally from 1 to 99%, preferably from 5 to 90%, more preferably from 10 to 80%. Similarly, in the present invention, the content of the pH dependent polymeric substances based on 1 part by weight of the non-pH dependent polymeric substances in the sustained-release portion is generally from 0.1 to 20 parts by weight, preferably from 0.2 to 10 parts by weight, more preferably from 0.3 to 5 parts by weight.

The composition according to the present invention is, of course, not limited to the above. The composition according to the present invention may be a composition in which dissolution control can be realized so as to achieve the effects of the anti-dementia drugs additively or synergistically, or so as to prevent or suppress the onset of the side effects, or with some other objective, this being in accordance with the structural characteristics and physicochemical characteristics of the anti-dementia drugs.

### EXAMPLES

The present invention is explained below in more detail below with reference to the following examples, but the present invention should not be construed as being limited thereto. The additives used in the pharmaceutical compositions were reagents, or additives complying with official documents such as the Japanese Pharmacopoeia 14^{th} Edition, the Japanese Pharmaceutical Excipients 2003 (JPE), and the Japan Pharmaceutical Codex 1997 (JPC).

### Example 1

6 g of donepezil hydrochloride (Eisai Co. Ltd.), 12 g of memantine hydrochloride (Lachema s.r.o.), 28.8 g of Ethocel 10FP (ethylcellulose, Dow Chemical), 36 g of Eudragit L100-55 (Röhm GmbH & Co. KG), and 45.6 g of lactose were mixed together in a granulator. An aqueous solution of 2.4 g of hydroxypropyl cellulose (HPC-L; Nippon Soda Co., Ltd) in a suitable amount of purified water was added to the mixture and wet granulation was carried out, and then the thus-obtained granules were heat dried using a tray dryer, and then sieved to obtain the desired granule size. After sizing, 1 g of magnesium stearate (Mallinckrodt Baker, Inc.) based on 109 g of granules was added and mixed in, and then a rotary tabletting machine was used to form tablets, whereby compression-molded products with diameter in 8 mm containing 10 mg of donepezil hydrochloride and 20 mg of memantine hydrochloride in a 220 mg tablet were obtained. Opadry yellow (Colorcon Japan Limited) was used to give the resulting products a water-soluble film coating containing hydroxypropyl methylcellulose as its main component (coating amount: 8 mg/tablet), resulting in film-coated tablets.

### Example 2

5 g of donepezil hydrochloride (Eisai Co. Ltd.), 10 g of memantine hydrochloride (Lachema s.r.o.), 20 g of corn starch (Nihon Shokuhin Kako Co., Ltd.), 15 g of crystalline cellulose (Asahi Kasei Corporation), and 81.75 g of lactose were mixed together in a granulator. An aqueous solution of 3.0 g of hydroxypropyl cellulose in a suitable amount of purified water was added to the mixture and wet granulation was carried out, and then the thus-obtained granules were heat dried using a tray dryer, and then sieved to obtain the desired granule size. After sizing, 0.25 g of magnesium stearate (Mallinckrodt Baker, Inc.) based on 134.75 g of granules was added and mixed in, and then a rotary tabletting machine was used to form tablets, whereby compression-molded products with diameter in 7 mm containing 5 mg of donepezil hydrochloride and 10 mg of memantine hydrochloride in a 135 mg tablet were obtained. Opadry yellow (Colorcon Japan Limited) was used to give the resulting products a water-soluble film coating containing hydroxypropyl methylcellulose as its main component (coating amount: 5 mg/tablet), resulting in film-coated tablets.

### Example 3

12 g of memantine hydrochloride (Lachema s.r.o.), 28.8 g of Ethocel 10FP (ethylcellulose, Dow Chemical Company), 36 g of Eudragit L100-55 (Röhm GmbH & Co. KG), and 39.6 g of lactose were mixed together in a granulator. An aqueous solution of 2.4 g of hydroxypropyl cellulose (HPC-L; Nippon Soda Co., Ltd) in a suitable amount of purified water was added to the mixture and wet granulation was carried out, and then the thus-obtained granules were heat dried using a tray dryer, and then sieved to obtain the desired granule size. After sizing, 1 g of magnesium stearate (Mallinckrodt Baker, Inc.) based on 99 g of granules was added and mixed in, and then a rotary tabletting machine was used to form tablets, whereby compression-molded products with diameter in 8 mm containing 20 mg of memantine hydrochloride in a 200 mg tablet were obtained. On the other hand, 3 g of donepezil hydrochloride (Eisai Co. Ltd.), 19.2 g of corn starch (Nihon Shokuhin Kako Co., Ltd.), 14.4 g of crystalline cellulose (Asahi Kasei Corporation), and 89.88 g of lactose were mixed together in a granulator. An aqueous solution of 2.88 g of hydroxypropyl cellulose (HPC-L; Nippon Soda Co., Ltd) in a suitable amount of purified water was added to the mixture and wet granulation was carried out, and then the thus-obtained granules were heat dried using a tray dryer, and then sieved to obtain the desired granule size. After sizing, 0.4 g of magnesium stearate (Mallinckrodt Baker, Inc.) based on 215.6 g of granules was added and mixed in, whereby a mixture containing donepezil hydrochloride was obtained. Subsequently, using 216 mg of the mixture containing donepezil hydrochloride based on one tablet of the compression-molded products containing memantine hydrochloride, a press-coated tabletting machine was used to form tablets, whereby press-coated tablets comprising an outer layer containing 5 mg of donepezil hydrochloride and an inner core layer containing 20 mg of memantine hydrochloride in a 416 mg tablet were obtained.

### Example 4

6 g of donepezil hydrochloride (Eisai Co. Ltd.), 28.8 g of Ethocel 10FP (ethylcellulose, Dow Chemical Company), 36 g of Eudragit L100-55 (Röhm GmbH & Co. KG), and 57.6 g of lactose were mixed together in a granulator. An aqueous solution of 2.4 g of hydroxypropyl cellulose (HPC-L; Nippon Soda Co., Ltd) in a suitable amount of purified water was added to the mixture and wet granulation was carried out, and then the thus-obtained granules were heat dried using a tray dryer, and then sieved to obtain the desired granule size. After sizing, 1 g of magnesium stearate based on 99 g of granules was added and mixed in, and then a rotary tabletting machine was used to form tablets, whereby compression-molded products with diameter in 8 mm containing 10 mg of donepezil hydrochloride in a 200 mg tablet were obtained. On the other hand, 6 g of memantine hydrochloride (Lachema s.r.o.), 19.2 g of corn starch (Nihon Shokuhin Kako Co., Ltd.), 14.4 g of crystalline cellulose (Asahi Kasei Corporation), and 86.88 g of lactose were mixed together in a granulator. An aqueous solution of 2.88 g of hydroxypropyl cellulose in a suitable amount of purified water was added to the mixture and wet granulation was carried out, and then the thus-obtained granules were heat dried using a tray dryer, and then sieved to obtain the desired granule size. After sizing, 0.4 g of magnesium stearate (Mallinckrodt Baker, Inc.) based on 215.6 g of granules was added and mixed in, whereby a mixture containing memantine hydrochloride was obtained. Subsequently, using 216 mg of the mixture containing memantine hydrochloride based on one tablet of the compression-molded products containing donepezil hydrochloride, a press-coated tabletting machine was used to form tablets, whereby dried-coated tablets comprising an outer layer containing 10 mg of memantine hydrochloride and an inner core layer containing 10 mg of donepezil hydrochloride in a 416 mg tablet were obtained.

The present invention is not limited to the specifically disclosed embodiments, and variations and modifications may be made without departing from the scope of the present invention.

The present application is based on Japanese priority patent application No. 2004-376770 filed on Dec. 27, 2004 and U.S. provisional patent application No. 60/675,483 filed on Apr. 28, 2005, the entire contents of which are hereby incorporated by reference.

What is claimed is:

1. A composition comprising at least two kinds of anti-dementia drugs.

2. The composition according to claim 1, further comprising at least one sustained-release portion comprising the anti-dementia drug.

3. The composition according to claim 1 or 2, further comprising at least one immediate-release portion comprising the anti-dementia drug.

4. The composition according to claim 2, wherein the sustained-release portion comprises at least one selected from an non-pH dependent polymeric substance and a pH dependent polymeric substance.

5. The composition according to claim 3, wherein the sustained-release portion comprises at least one selected from an non-pH dependent polymeric substance and a pH dependent polymeric substance.

6. The composition according to claim 1, wherein the anti-dementia drugs comprise a combination of a cholinesterase inhibitor and a compound having a mechanism of action different from that of the cholinesterase inhibitor.

7. The composition according to claim 1, wherein the anti-dementia drugs comprise at least one cholinesterase inhibitor and at least one N-methyl-D-aspartate receptor antagonist.

8. The composition according to claim 1, wherein the anti-dementia drugs comprise donepezil or a pharmacologically acceptable salt thereof, and memantine or a pharmacologically acceptable salt thereof.

9. The composition according to claim 2, wherein the anti-dementia drug contained in the sustained-release portion is memantine hydrochloride.

10. The composition according to claim 3, wherein the anti-dementia drug contained in the sustained-release portion is memantine hydrochloride, and the anti-dementia drug contained in the immediate-release portion is donepezil hydrochloride.

11. The composition according to claim 2, further comprising two of the sustained-release portions, wherein one sustained-release portion comprises memantine hydrochloride as the anti-dementia drug, and another sustained-release portion comprises donepezil hydrochloride as the anti-dementia drug.

12. The composition according to claim 2, wherein memantine or a pharmacologically acceptable salt thereof and donepezil or a pharmacologically acceptable salt thereof are contained in said one sustained-release portion.

13. The composition according to claim 3, wherein memantine or a pharmacologically acceptable salt thereof and donepezil or a pharmacologically acceptable salt thereof are contained in said one immediate-release portion.

14. The composition according to claim 4, wherein the non-pH dependent polymeric substance comprises a water-insoluble polymeric substance.

15. The composition according to claim 4, wherein the pH dependent polymer comprises an enteric polymeric substance.

16. The composition according to claim 5, wherein the non-pH dependent polymeric substance comprises a water-insoluble polymeric substance.

17. The composition according to claim 5, wherein the pH dependent polymer comprises an enteric polymeric substance.

18. The composition according to claim 2, wherein the sustained-release portion comprises a granule or a compression-molded product.
